(19) United States
(12) Patent Application Publication
(10) Pub. No.: US 2011/0027846 A1
(43) Pub. Date: Feb. 3, 2011

(54) CONTROL OF BACTERIA IN FERMENTATION PROCESSES

(76) Inventors: José Sebastiao De Sa, Sao Bernardo do Campo (BR); Abel Oliveira, Sao Paulo City (BR)

Correspondence Address: The Dow Chemical Company, P.O. BOX 1967, 2040 Dow Center, Midland, MI, 48641, US

(21) Appl. No.: 12/666,839

(22) PCT Filed: Jun. 24, 2008
(86) PCT No.: PCT/IB08/01651

§ 371 Date: Jun. 9, 2010

Related U.S. Application Data
(60) Provisional application No. 60/937,500, filed on Jun. 28, 2007.

(51) Int. Cl. C12P 7/06

(52) U.S. Cl. 435/161

(57) ABSTRACT

A method of producing a fermentation-based product comprises fermenting a sugar-containing medium with yeast in the presence of an organic biocide and a quaternary ammonium compound, in amounts sufficient to reduce or control a bacterial population in the sugar-containing medium. The additives enable reduction or elimination of antibiotics while still showing desirably reduced percent infection, process variability and interference with yeast viability.

### BACKGROUND OF THE INVENTION

1. Technical Field

This invention relates to the field of fermentation processes. More particularly, it relates to methods and means for controlling bacteria in fermentation processes for producing ethanol.

2. Background of the Art

A commonly-employed method of producing ethanol involves fermentation based on yeast. This process consists basically of the following operations: (a) molasses handling, dilution, clarification and heat treatment, (b) anaerobic fermentation by a selected yeast strain, previously grown under controlled conditions, (c) yeast separation from the broth, and (d) alcohol separation by distillation and eventual storage thereof. For detailed descriptions and technical details, see, for example: Harrison, J. S, and Graham, J. C. J., “Yeast in Distillery Practice” in A. H. Rose and J. S. Harrison (Eds.) “The Yeasts” 3 (6) 283-348 (1970), Academic Press; Kampen, W. H., Sugar y Azucar 70 (8) 36-39, 42-43 (1975); and L′Anson, J. A. P., Process Biochem. 11 (7) 35-39 (1971). Many processing operations begin with the juice or syrup which has been extracted from a solid fiber matrix of a sugar source selected from, for example, sugar cane, corn, or sugar beets, while others begin with direct fermentation of the sugar source which has been comminuted into fragments or highly pulverized. Such methods represent generally efficient ways to produce a variety of alcohols, and in particular ethanol, from a selected fermentation substrate.

However, a problem is encountered when bacteria contaminate the fermentation substrate. The bacteria, when present at relatively high levels, compete with the yeast and may reduce the fermentative yield. Furthermore, the bacteria may cause flocculation, requiring additional measures to obtain ethanol therefrom. Those skilled in the art have developed various means of addressing the bacteria problem. The most commonly used method at present involves adding biocides to the substrate. Examples of these biocides include quaternary ammonium compounds, carbamates, and halogenated phenols. Alternatively or in combination with biocides, hydrogen peroxide or antibiotics may be used. Such may include, for example, an antibiotic known as KAMORAN HJ™, which is defined as 4-[2-[5-ethyl-5-[5-[6-hydroxy-6-(hydroxymethyl)-3,5-dimethyl-oxan-2-y]-3-methyl-oxolan-2-yl]oxolan-2-yl]-9-hydroxy-2,8-dimethyl-1,6-dioxaspiro[4.5]dec-7-yl]-3-methoxy-2-methyl-pentanoate.

Unfortunately, some biocides and antibiotics may undesirably contaminate the ethanol, cause flocculation, or require a post-treatment or additional processing of the fermentation medium and/or the alcohol product. Such post-treatments or additional processing may add to the time, cost, and/or convenience of producing the ethanol. Biocides and antibiotics may also reduce yeast level during the process, which is undesirable.

In view of the above, it would be desirable in the art to find methods and means for preparing ethanol via fermentation processes that eliminate or reduce these problems and/or the need for biocides and antibiotics conventionally used for such processes.

### SUMMARY OF THE INVENTION

Accordingly, the present invention provides, in one aspect, a method of producing fermentation-based products, particularly ethanol, comprising fermenting a sugar-containing medium with yeast in the presence of an organic biocide and a quaternary ammonium compound, in amounts sufficient to reduce or control a bacterial population in the sugar-containing medium. In certain non-limiting embodiments the organic biocide is selected from the group consisting of aliphatic and aromatic monoaldehydes and dialdehydes; carbamates; halogenated and non-halogenated phenolics, and their corresponding sodium and potassium salts; compounds that release formaldehyde upon contact with water; guanidine-based compounds; isothiazolinone compounds; 2-bromo-2-nitro-1,3-propanediol (“Bronopol”); bromonitrostyrene; 2,2-dibromo-3-nitrilopropionamide (DBNPA); 2,6-dimethyl-m-dioxan-4-ol acetate; and combinations thereof.

In another aspect, the invention provides a method of producing ethanol comprising fermenting a sugar-containing medium with yeast in the presence of glutaraldehyde and a quaternary ammonium compound, in amounts sufficient to reduce or control a bacterial population in the sugar-containing medium.

### BRIEF DESCRIPTION OF THE DRAWINGS

FIG. 1 shows a comparison of yeast flocculation levels for a conventional biocide/antibiotic combination and the inventive treatment.

FIG. 2 shows a comparison of bacterial contamination levels for a conventional biocide/antibiotic combination and the inventive treatment.

FIG. 3 shows a comparison of infection percent for a conventional biocide/antibiotic combination and the inventive treatment.

FIG. 4 shows a comparison of yeast viability percent for a conventional biocide/antibiotic combination and the inventive treatment.

### DETAILED DESCRIPTION OF THE INVENTION

The present invention is a method for carrying out ethanol production from a variety of sugar-containing sources, including, but not limited to, sugar cane, corn, sugar beets, cellulosic feedstocks, date palm, sorghum, sugar maple, combinations thereof, and the like. “Sugar,” as used herein, refers to any chemically-defined sugar, i.e., a monosaccharide, disaccharide, trisaccharide, or oligosaccharide, that is suitable to be fermented to produce a fermentation product, and in particular, ethanol.

Preparation of the Sugar-Containing Medium Used for Fermentation is Well known to those skilled in the art, and generally includes either extraction of a juice via crushing of the sugar-containing source and/or of the seeds thereof. Recovery of sucrose from the cane plant requires the separation of juice from the fibrous material in the structure of the stalk. The tissue inside the rind of the stalk is a matrix of thin-walled parenchyma storage cells in which vascular bundles are imbedded. This parenchymatous tissue is called the “pith,” while the rind and the vascular bundles are collectively referred to as the “fiber.” Sucrose is present principally in the parenchyma storage cells. These cells are easily ruptured, and the most commonly employed methods to extract the juice are milling or crushing, called grinding; hot water extraction, or “diffusion”; or a combination of both methods. During grinding, hot water is sprayed over the shredded material to extract any remaining sugar and add it to the raw juice. In the diffusion method, the cane is prepared by a combination of knife mills and roller crushers. The solid waste remaining after extraction of the sugar is known as pulp or sugar cane bagasse, which is separated out and dried for use as fuel.

The raw juice is then heated and spun in a centrifuge, whereby a thick syrup is forced out through small holes in the centrifuge walls. This syrup is called molasses, which has its own uses, such as in commercial table syrup and animal feed. The remaining material, a solid, is then sent to a refinery. Here it is redissolved and decolorized, and may then be either recrystallized into a desired size, or used to prepare a fermentation substrate, as in the case in the present invention.

In the case of some materials, such as sugar cane and cellulosic feedstocks, additional pre-fermentation steps may be required, such as enzyme or acid cleavage to break glycosidic bonds, and the like. Similar methods are typically used for extraction and preparation of sugar from the other sugar-containing sources, but those skilled in the art will understand that any method may be employed in the practice of the invention, provided that the result is a sugar-containing source in a form that is useful for preparation of a sugar-containing medium for fermentation, i.e., a fermentation substrate, which is an aqueous suspension of the sugar. The amount of water is desirably based upon the amount of sugar, as is well known to those skilled in the art. In general, too much water may be undesirable because it will dilute the final ethanol concentration, hence increasing the energy demand for purification, while too little water will not produce an adequate suspension.

In addition to the sugar and water, the fermentation substrate will include yeast. The particular yeast inoculum employed in the practice of the present invention is not considered to be critical. Illustrative yeast strains useful in the practice of the invention are those maintained at, for example, the Central American Research Institute for Industry, Avenida La Reforma 4-47, Zone 10, Guatemala, C.A. (Instituto Centroamericano de Investigacion y Tecnologia Industrial, “ICAITI”) as strains Saccharomyces cerevisae L-180; Saccharomyces cerevisae L-181; Saccharomyces L-200; Saccharomyces L-208; Saccharomyces L-140; and Saccharomyces cerevisae L-169 (hybrid 5-non-flocculant). In some non-limiting embodiments preference may be given to Saccharomyces cerevisae strains L-180 and L-181, which are also deposited at the Central Bureau Voor Schimmel Culture, Delft, Holland, under the strain numbers CBS 2959 and CBS 1242, respectively.

Fermentation may be carried out over any desired time period in which a desired amount of fermentation-based products are produced. Such may range, in one non-limiting embodiment, from one day to six months. In another non-limiting embodiment, the time period may range from one day to two months.

Those skilled in the art will be aware of appropriate equipment, including tanks, vats, and the like for carrying out the process. Because fermentation of sugar-containing media produces, among its fermentation products, carbon dioxide, it is necessary to ensure that appropriate means for channeling the carbon dioxide away from the sugar-containing medium are provided, to ensure that bursting of the medium container does not occur. One such approach is simply to conduct the fermentation in an open vessel. Other means include, for example, tubing or piping, in a closed vessel, above the surface of the medium, leading to an appropriate outlet.

The present invention includes carrying out at least a portion of the fermentation process in the presence of two additives, which are an organic biocide and a quaternary ammonium compound. These additives may be incorporated at any appropriate point in the process, which is generally performed as a batch operation. In certain non-limiting embodiments the organic biocide and quaternary ammonium compound are each added near or at the beginning of the fermentation process in the fermentation tank, and in other non-limiting embodiments they are added to the must tank. In still other non-limiting embodiments, one of the additives may be injected or otherwise introduced into the fermentation tank and the other added to the must tank. The two may alternatively be added separately to a single tank, or may be first blended together and added as a blend.

The organic biocide may be any organic compound having a range of from about 1 to about 20 carbon atoms, in certain non-limiting embodiments from about 5 to about 15 carbon atoms, that is known or found to be effective as a biocide in the given fermentation system, and which does not contain a quaternary ammonium functionality. In certain non-limiting embodiments the biocide is selected from the group consisting of aliphatic and aromatic monoaldehydes and dialdehydes, such as formaldehyde, glutaraldehyde, orthophthalic aldehyde, hexanedial, heptanedial, octanedial, hexanal, heptanal, and octanal. It may be selected from a halogenated or non-halogenated phenolic, such as o-phenylphenol or one of its corresponding sodium or potassium salts. It may be a carbamate, or a compound that releases formaldehyde upon contact with water, such as tetrakis(hydroxymethyl) phosphonium sulphate, an oxazolidine, a triazine, a hydantoin, cis/trans 1-(3-chloro-allyl)-3,5,7-triaza-1-azoniaadamantane chloride, or tris(hydroxymethyl)-nitro-methane. It may be selected from guanidine-based compounds, such as guanidine, biguanides, and polyguanides including, for example, polyhexamethylene biguanide hydrochloride (PHMB). It may be selected from isothiazolinone compounds, such as 5-chloro-2-methyl-4-isothiazolin-3-one, 2-methyl-4-isothiazo-lin-3-one, and 2-benzisothiazolin-3-one. It may be 2-bromo-2-nitro-1,3-propanediol (“Bronopol”), 2,2-dibromo-3-nitrilo-propionamide (DBNPA), bromonitrostyrene, or 2,6-dimethyl-m-dioxan-4-ol acetate. It may be a combination of two or more of any of the foregoing.

Commercially-available examples of oxazolidine compounds may further include, for example, DOWICIL™ 96 and BIOBAN™ CS-1135 from The Dow Chemical Company. Examples of triazines may include GROTAN™ from Troy Corporation. A commercially-available example of a hydantoin compound may include Dantogard™ from Lonza. A commercially-available example of cis/trans 1-(3-chloro-allyl)-3,5,7-triaza-1-azoniaadamantane chloride may include DOWICIL™ 75, available from The Dow Chemical Company. A commercially-available example of tris(hydroxymethyl)nitro-methane methane may include TRIS NITRO™ available from The Dow Chemical Company. Tetrakis (hydroxymethyl) phosphonium sulphate is available as AQUCAR™ THPS 75 from The Dow Chemical Company). The isothiazolinone compounds may include, for example, 5-chloro-2-methyl-4-isothiazolin-3-one with 2-methyl-4-isothiazolin-3-one, available as CANGUARD™ CM, and 2-benzisothiazolin-3-one, available as CANGUARD™ BIT, both from The Dow Chemical Company. Non-halogenated phenolics may include, for example, o-phenylphenol and its corresponding sodium and/or potassium salts, such as DOWICIDE™ manufactured by The Dow Chemical Company. Combinations of any of the additives and/or types of additives listed hereinabove may alternatively be selected.

In a particular non-limiting embodiment the organic biocide is glutaraldehyde, which has the general formula C₅H₈O₂ and the general structure

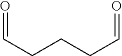

It is also called pentanedial or 1,5-pentanedione and may be obtained from a variety of commercial sources. Those skilled in the art will be aware of the many ways it may be prepared, including, for example, by conversion of a propylene feedstream to a heterodiene acrolein, followed by reaction of the acrolein with a vinyl ether to form 2-methoxy-3,4-dihydro-2H-pyran. The 2-methoxy-3,4-dihydro-2H-pyran may then be hydrolyzed in the presence of a suitable catalyst to produce glutaraldehyde. See, for example, U.S. Pat. No. 6,187,963. Other methods of preparing glutaraldehyde are well known, and those skilled in the art will be easily able to determine appropriate preparation steps. Glutaraldehyde is available commercially in various solution concentrates ranging from 1 percent to 50 percent by weight. Examples of such commercial glutaraldehyde solutions include those which are sold under the UCARCIDE™ tradename by The Dow Chemical Company.

The other category of additive in the present invention is the quaternary ammonium compound. Those skilled in the art will be familiar with the general structure for quaternary ammonium compounds, which is

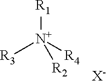

wherein each of R¹, R², R³ and R⁴ is selected from the group consisting of saturated and unsaturated alkyl, aryl, alkylaryl, phenyl, allyl, and alkylphenyl groups which may be connected together in any combination; and X is any halogen. These are salts having quaternary ammonium cations and halogen anions, wherein the cations remain permanently charged independent of the pH of their solution. They may generally be prepared by alkylation of ammonia or other amines, by treatment of an amine with a strong base which induces the so-called Hofmann Elimination, or by nucleophilic substitution of a tertiary amine with a haloalkane. Suitable non-limiting examples of quaternary ammonium compounds useful in the present invention include, but are not limited to, alkyl dimethyl benzyl ammonium chloride; cetyltrimethylammonium bromide; di-n-decyl-dimethylammonium chloride; dioctyl-dimethylammonium chloride; diallyl-dimethylammonium chloride; cetylpyridinium chloride; benzethonium chloride; and combinations thereof. Polymeric quaternary ammonium salts and mixtures of one or more quaternary ammonium compounds are also suitable for use in the present invention.

In general, the combination of both the organic biocide and the quaternary ammonium compound may range, in certain non-limiting embodiments, from about 10,000 parts per billion (ppb) to about 100 parts per million (ppm), and in other non-limiting embodiments may range from about 1 ppm to about 100 ppm, based on the total amount of fermentation substrate by weight. In still other non-limiting embodiments the combined amount may range from about 5 ppm to about 50 ppm, and in yet other non-limiting embodiments it may range from about 10 ppm to about 40 ppm. The relative proportions of the organic biocide to the quaternary ammonium compound may, in certain non-limiting embodiments, range from about 50:50 to about 95:5 by weight; in other non-limiting embodiments it may range from about 60:40 to about 90:10; in still other non-limiting embodiments it may range from about 70:30 to about 90:10; and in yet other non-limiting embodiments it may range from about 80:20 to about 90:10. In one particularly desirable embodiment it may be about 85:15 (all by weight).

Prior to beginning the fermentation procedure, the temperature of the aqueous, sugar-containing medium is, in many embodiments, elevated to within the range of from about 95° C. to about 105° C., that is, to approximately the boiling temperature of the water, for a relatively short period of from about 5 minutes to about 10 minutes. This serves to pasteurize the starting fermentation substrate. The substrate is then cooled, to a temperature most desirably within the range of about 28° C. to about 35° C., at which temperature an inoculum of the selected fermentation microorganism (yeast) is introduced into the aqueous suspension.

The starting proportions of water:sugar:yeast may be varied according to the knowledge of those skilled in the art and optimized in a given production situation on the basis of routine experimentation. However, in one non-limiting embodiment it has been found that a water:sugar:yeast weight proportionality of 78:15:7 may be effective. In general, a water:sugar:yeast weight proportion of from about 70:20:10 to about 80:15:5 may be desirable, though use of more or less of each proportion may be effective. However, such proportional variations will obviously affect total fermentation time and/or yield under identical conditions. The organic biocide and quaternary ammonium compound combination may be added at any time, but preferably with the addition of the water, sugar and yeast. When the organic biocide is glutaraldehyde, the combination is preferably added either before the fermentation step and/or during the fermentation step, including simultaneously to the sugar-cane juice and yeast feeding procedure. In the invention, it is not necessary to deactivate the glutaraldehyde before addition of the yeast, and in fact it is an advantage of the invention that the glutaraldehyde can be present throughout the fermentation reaction without detrimental effect on the reaction.

The pH of the suspension may then be adjusted to within a range of from about 1.5 to 7 employing, for example, hydrochloric acid or other standard reagent for this purpose, usually contemporaneously with addition of the yeast, in order to provide optimum conditions for effective fermentation. A separate source of inorganic nitrogen may be added in order to increase conversion to ethanol, but such may not be necessary in all methods of production of fermentation products. See, for example, Bose, K. and Ghose, T. K., Process Biochem. 8 (2) 23 (1973), which is incorporated herein by reference in its entirety.

During the fermentation the sugar, for example, sucrose, in the sugar-containing medium will be transformed by the yeast into ethanol and carbon dioxide, on a stoichiometric basis, under anaerobic conditions. This sugar consumption will tend to decrease the bulk concentration of sugar in solution. Also as fermentation proceeds, the ethanol bulk concentration in solution will increase.

Once the fermentation is complete and all the sugar has been, as a consequence, converted to ethanol and carbon dioxide, the ethanol may be recovered. Such may be accomplished by any means known to those skilled in the art, for example, by standard filtration and distillation of the ethanol/yeast suspension. The ethanol so recovered is useful for many industrial purposes and commercial purposes.

The final result may be, in certain non-limiting embodiments, fermentation product production wherein is experienced a lower bacteria contamination level, better process control (reduced variability of bacteria contamination level), reduced interference with yeast viability, reduced yeast flocculation, reduced process infection level, and reduced or eliminated need for antibiotics. The term “process infection level” refers to the ratio between total bacteria level to yeast level. The lower the number, the better the overall process efficiency.

The description and examples discussed hereinabove and below are intended to provide to the skilled practitioner the general concepts, means and methods necessary to understand the invention and, when combined with a level of understanding typical of those skilled in the art, to practice it. It will therefore be understood that not all embodiments deemed to be within the scope of the invention are herein explicitly described, and that many variations of each embodiment, including but not limited to selection of the sugar source, specific biocides and quaternary ammonium compounds, addition point and addition order, fermentation and other processing times, temperatures and other conditions, treatment protocols and equipment, and the like, not described explicitly or in detail herein, will still fall within the general scope of the invention.

The invention having been generally described, the following examples are given as particular embodiments of the invention and to demonstrate the practice and advantages thereof. It is understood that the examples are given by way of illustration and are not intended to limit the specification or the claims to follow in any manner. Unless specified otherwise, all amounts here are by weight.

### EXAMPLES

### Comparative Example 1

This example illustrates application of the invention in a trial using an industrial scale fermentation tank. In this example the process infection level is measured in the fermentation tank on a daily basis. The example compares use of a conventional treatment, in which a quaternary ammonium compound is used along with an antibiotic for the first 15 days, and then an organic biocide/quaternary ammonium compound combination, denominated as BIOBAN™ ETH 1000, is employed for the next 20 days. The BIOBAN™ ETH 1000 product is a 50 percent active product containing glutaraldehyde as the organic biocide, and alkyl dimethyl benzyl ammonium chloride as the quaternary ammonium compound, together in a weight ratio of 85:15. Both the conventional treatment and the BIOBAN™ ETH 1000 are used in total amounts of about 30 ppm by weight. The fermentation tank contains the sugar-containing medium, which is a suspension of water, sugar obtained from sugar cane, and yeast (Saccharomyces cerevisae) in weight proportions of water:sugar:yeast of 78:15:7, as measured by weight.

This example shows a substantial overall reduction in percent infection when the inventive combination of additives is employed. It also shows that process control is improved with the invention, in that there is reduced variability of the percent infection with the use of the BIOBAN™ ETH 1000. The results are shown in Table 1.

TABLE 1

| Day | % Infection | System With or Without BIOBAN ™ ETH 1000 |
|---|---|---|
| 2 | 0.72 | Without* |
| 3 | 2.19 | Without* |
| 4 | 20.22 | Without* |
| 5 | 29.28 | Without* |
| 6 | 15.13 | Without* |
| 7 | 15.13 | Without* |
| 8 | 6.45 | Without* |
| 9 | 5.56 | Without* |
| 10 | 6.28 | Without* |
| 11 | 9.79 | Without* |
| 12 | 27.50 | Without* |
| 13 | 11.02 | Without* |
| 14 | 1.40 | Without* |
| 15 | 2.34 | With |
| 16 | 4.27 | With |
| 17 | 0.60 | With |
| 18 | 2.03 | With |
| 19 | 3.63 | With |
| 20 | 2.57 | With |
| 21 | 1.17 | With |
| 22 | 4.03 | With |
| 23 | 1.14 | With |
| 24 | 0.62 | With |
| 25 | 1.00 | With |
| 26 | 1.62 | With |
| 27 | 5.49 | With |
| 28 | 0.63 | With |
| 29 | 1.62 | With |
| 30 | 5.49 | With |
| 31 | 0.63 | With |
| 32 | 1.62 | With |
| 33 | 3.63 | With |
| 34 | 2.01 | With |

*indicates not an example of the invention.

### Comparative Example 2

This example shows that yeast viability is maintained with use of the additive combination at a level comparable to that when the conventional combination of biocide and antibiotics, as used in Comparative Example 1, is employed. In this comparative example the indicated biocides are used at what is considered in the art to be a minimal level, while the antibiotic level varies depending upon the severity of the contamination with other microorganisms. Each biocide is added before or at the early fermentation stage, and the antibiotic is added during the fermentation as required. Samples are obtained from the same industrial scale fermentation system as described in Example 1, but the period in which the biocide/antibiotic combination is used extends to Day 25, and the subsequent period wherein BIOBAN™ ETH 1000 is used instead extends from Day 26 to Day 51. In both cases the total level of the treatment agent is maintained at about 40 ppm, but the BIOBAN™ ETH 1000 is added to the must tank, while the conventional biocide/antibiotic combination is employed in the fermentation tank. The results are shown in Table 2.

TABLE 2

| | % Yeast | |
|---|---|---|
| Day | Viability | System With or Without BIOBAN ™ ETH 1000 |
| 2 | 83.66 | Without* |
| 3 | 84.36 | Without* |
| 4 | 85.33 | Without* |
| 5 | 81.77 | Without* |
| 6 | 88.79 | Without* |
| 7 | 83.33 | Without* |
| 8 | 90.85 | Without* |
| 9 | 88.84 | Without* |
| 10 | 58.33 | Without* |
| 11 | 79.59 | Without* |
| 12 | 83.80 | Without* |
| 13 | 67.95 | Without* |
| 14 | 82.86 | Without* |
| 15 | 87.13 | Without* |
| 16 | 86.86 | Without* |
| 17 | 84.66 | Without* |
| 18 | 80.46 | Without* |
| 19 | 84.33 | Without* |
| 20 | 85.59 | Without* |
| 21 | 80.00 | Without* |
| 22 | 81.88 | Without* |
| 23 | 79.59 | Without* |
| 24 | 87.57 | Without* |
| 25 | 73.97 | Without* |
| 26 | 81.32 | With |
| 27 | 77.81 | With |
| 28 | 73.61 | With |
| 29 | 81.13 | With |
| 30 | 78.86 | With |
| 31 | 73.30 | With |
| 32 | 74.35 | With |
| 33 | 80.49 | With |
| 34 | 80.43 | With |
| 35 | 74.19 | With |
| 36 | 76.84 | With |
| 37 | 86.00 | With |
| 38 | 82.60 | With |
| 39 | 81.71 | With |
| 40 | 84.79 | With |
| 41 | 80.11 | With |
| 42 | 86.44 | With |
| 43 | 84.94 | With |
| 44 | 88.34 | With |
| 45 | 79.11 | With |
| 46 | 77.92 | With |
| 47 | 82.21 | With |
| 48 | 83.44 | With |
| 49 | 83.59 | With |
| 50 | 88.00 | With |
| 51 | 85.84 | With |

*indicates not an example of the invention.

### Comparative Example 3

A comparison is done to show the effect of four different biocides on yeast level and bacteria level in various media and at various points relative to introduction of the biocide. SDA medium is synthetic defined agar, and PCA medium is plate count agar. Dow Antimicrobial™ 7287 is a composition containing 20 percent 2,2-dibromo-3-nitrilopropionamide (DBNPA), sold by The Dow Chemical Company. The results are shown in Table 3.

TABLE 3

| | Biocide | Yeast | Bacteria | | Bacteria |
|---|---|---|---|---|---|
| | concentration | level¹ | level² | Yeast level³ | level⁴ |
| Biocide | (ppm) | (cfu/ml)* | (cfu/ml) | (cfu/ml) | (cfu/ml) |
| BIOBAN ™ ETH | 50 | | | 1.3 × 10⁵ | 6.0 × 10⁵ |
| 1000 | | | | | |
| Diethyl | 50 | | | <1.0 × 10⁴ | 2.6 × 10⁷ |
| Carbamate** | | | | | |
| Dow | 50 | | | 8.0 × 10⁴ | 4.0 × 10⁵ |
| Antimicrobial⁵ | | | | | |
| 7287 ™** | | | | | |
| UCARCIDE ™ | 50 | | | 5.0 × 10⁴ | 5.0 × 10⁵ |
| 250 | | | | | |
| Control** | 0 | 2.0 × 10⁴ | 8.4 × 10⁷ | 1.2 × 10⁴ | 3.3 × 10⁸ |

*cfu/ml is colony forming units per milliliter
**indicates not an example of the invention
¹denotes SDA medium
²denotes PCA medium
³denotes SDA medium, 2 hours after contact
⁴denotes PCT medium, 2 hours after contact
⁵denotes precipitation and color change of the fermentation medium were seen

### Comparative Example 4

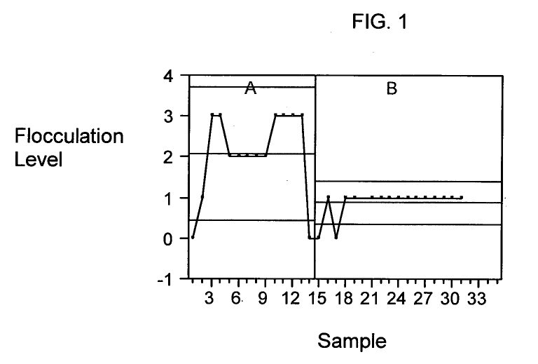

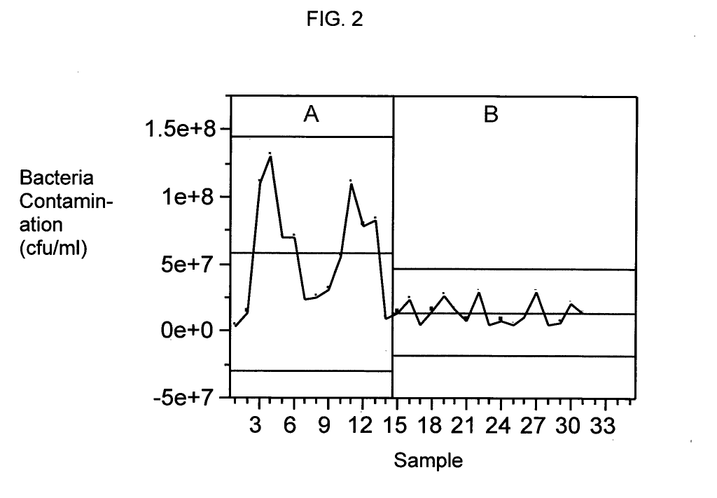

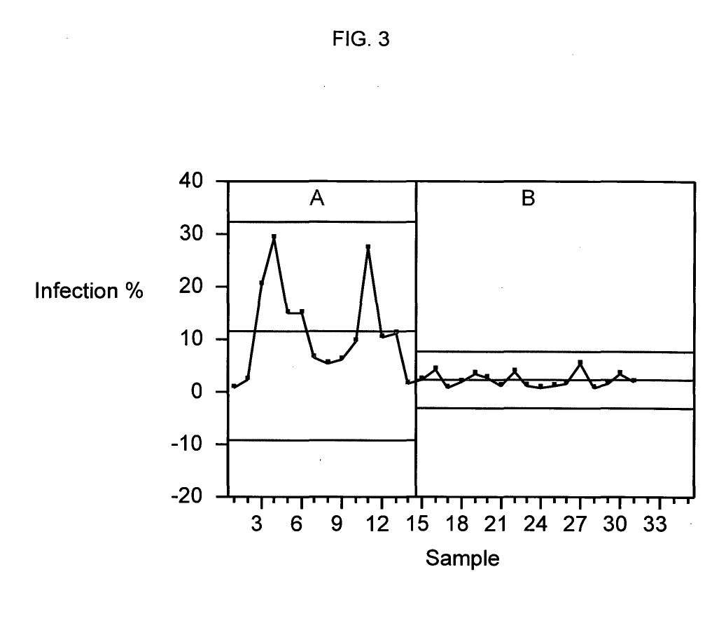

A comparison is done to show performance of the inventive process with a conventional biocide and antibiotic combination in a commercial scale fermentation plant. The yeast is Saccharomyces cerevisae, which is added to the must tank. Period “A” represents the first 15 days, during which the conventional biocide and antibiotic combination are used, and Period “B” represents the next 20 days, during which the inventive process and additives are employed. Sugar level is kept constant at 18° Bx (“degrees brix”), which is 18 grams of sucrose per 100 grams of liquid. The invention shows substantial improvements in yeast flocculation (FIG. 1), bacterial contamination (FIG. 2), and infection percent (FIG. 3) under the inventive process.

The data in FIGS. 1-3 shows that BIOBAN™ ETH 1000 in the range of from about 30 ppm to about 40 ppm is highly effective for the control of bacterial growth during the production of ethanol from sugar cane. It also shows that the inventive process is generally stabilized, thus reducing the need for plant shutdowns and expensive cleanouts. Finally, this comparative example strongly suggests that the invention may eliminate the need to use antibiotics or other biocides to control the system when either microbiologic (e.g., infection percent and bacteria contamination level) or processes parameters (e.g., yeast flocculation) get out of control.

### Comparative Example 5

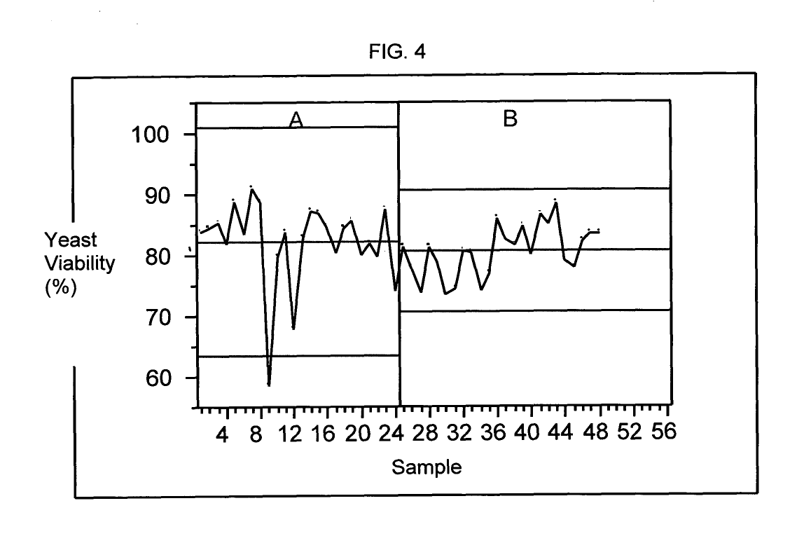

Yeast viability is tested via a comparative industrial scale trial. In this trial a quaternary ammonium compound is used during the first 24 days, along with antibiotic as needed (Period “A”). During Period “B”, BIOBAN™ ETH 1000 is used. It will be seen in FIG. 4 that yeast viability is better maintained with the BIOBAN™ ETH 1000.

What is claimed is:

1. A method of producing a fermentation-based product comprising fermenting a sugar-containing medium with yeast in the presence of an organic biocide and a quaternary ammonium compound, in amounts sufficient to reduce or control a bacterial population in the sugar-containing medium.

2. The method of claim 1 wherein the fermentation-based product is ethanol.

3. The method of claim 1 wherein the sugar-containing medium includes sugar obtained from the group consisting of sugar cane, sugar beets, date palm, sorghum, sugar maple, corn, cellulosic feedstocks, and combinations thereof.

4. The method of claim 1 wherein the organic biocide is selected from the group consisting of compounds having from 1 to 20 carbon atoms.

5. The method of claim 4 wherein the organic biocide is selected from the group consisting of aliphatic and aromatic monoaldehydes and dialdehydes;
carbamates; halogenated and non-halogenated phenolics and their corresponding sodium and potassium salts; compounds that release formaldehyde upon contact with water; guanidine-based compounds; isothiazolinone compounds; 2-bromo-2-nitro-1,3-propanediol (“Bronopol”); bromonitrostyrene; 2,2-dibromo-3-nitrilopropion-amide (DBNPA); 2,6-dimethyl-m-dioxan-4-ol acetate; and combinations thereof;

6. The method of claim 5 wherein the organic biocide is selected from the group consisting of formaldehyde; glutaraldehyde; orthophthalic aldehyde; hexanedial; heptanedial; octanedial; hexanal; heptanal; octanal; o-phenylphenol and its corresponding sodium and potassium salts; tetrakis(hydroxymethyl) phosphonium sulphate; oxazolidines; triazines; hydantoins; cis/trans 1-(3-chloro-allyl)3,4,7-triaza-1-azoniaadamantane chloride; tris(hydroxymethyl)nitro-methane; guanidine; biguanides; polyguanides; 5-chloro-2-methyl-4-isothiazolin-3-one; 2-methyl-4-isothiazolin-3-one; 2-benzisothiazolin-3-one; and combinations thereof.

7. The method of claim 1 wherein the quaternary ammonium compound is selected from the group consisting of alkyl dimethyl benzyl ammonium chloride; cetyltrimethylammonium bromide; di-n-decyl-dimethylammonium chloride; dioctyl-dimethylammonium chloride; diallyl-dimethylammonium chloride; cetylpyridinium chloride; benzethonium chloride; polymeric quaternary ammonium salts; and combinations thereof.

8. The method of claim 1 wherein the amount of the organic biocide and the quaternary ammonium compound, combined, ranges from about 10,000 ppb to about 100 ppm.

9. The method of claim 8 wherein the amount of the organic biocide and the quaternary ammonium compound, combined, ranges from about 1 ppm to about 50 ppm.

10. The method of claim 8 wherein the organic biocide and the quaternary ammonium compound are used in a proportion of from about 50:50 to about 95:5.

11. The method of claim 10 wherein the organic biocide and the quaternary ammonium compound are used in a proportion of from about 80:20 to about 90:10.

12. The method of claim 10 wherein the organic biocide is glutaraldehyde and the quaternary ammonium compound is alkyl dimethyl benzyl ammonium chloride.

13. A method of producing ethanol comprising fermenting a sugar-containing-medium with yeast in the presence of glutaraldehyde and a quaternary ammonium compound, in amounts sufficient to reduce or control a bacterial population in the sugar-containing medium.

14. The method of claim 13 wherein the quaternary ammonium compound is selected from the group consisting of alkyl dimethyl benzyl ammonium chloride, cetyltrimethylammonium bromide, di-n-decyl-dimethylammonium chloride, dioctyl-dimethylammonium chloride, diallyl-dimethylammonium chloride, cetylpyridinium chloride, benzethonium chloride, polymeric quaternary ammonium salts, and combinations thereof.

15. The method of claim 13 wherein the amount of the glutaraldehyde and the quaternary ammonium compound, combined, ranges from about 10,000 ppb to about 100 ppm.

16. The method of claim 15 wherein the amount of the organic biocide and the quaternary ammonium compound, combined, ranges from about 1 ppm to about 50 ppm.

17. The method of claim 13 wherein the glutaraldehyde and the quaternary ammonium compound are used in a proportion of from about 50:50 to about 95:5.

18. The method of claim 17 wherein the glutaraldehyde and the quaternary ammonium compound are used in a proportion of from about 80:20 to about 90:10.

19. The method of claim 13 wherein the glutaraldehyde and the quaternary ammonium compound are added to the sugar cane medium in a fermentation tank, a must tank, or a combination thereof.

20. The method of claim 1 wherein antibiotics are not used or are used in an amount that is less than would be needed to result in an equivalent reduction or control of the bacterial population absent the organic biocide and the quaternary ammonium compound.
